(19) United States
(12) Patent Application Publication
(10) Pub. No.: US 2013/0193339 A1
(43) Pub. Date: Aug. 1, 2013

(54) RADIATION IRRADIATION INITIATION DETERMINATION APPARATUS, RADIATION IMAGE CAPTURING DEVICE, RADIATION IMAGE CAPTURE CONTROL APPARATUS, RADIATION IRRADIATION INITIATION DETERMINATION METHOD, AND COMPUTER READABLE MEDIUM

(71) Applicant: Fujifilm Corporation, Tokyo (JP)

(72) Inventor: Yasufumi ODA, Ashigarakami-gun (JP)

(73) Assignee: Fujifilm Corporation, Tokyo (JP)

(21) Appl. No.: 13/720,838

(22) Filed: Dec. 19, 2012

(30) Foreign Application Priority Data
Jan. 30, 2012 (JP) 2012-016682

(51) Int. Cl. G01T 1/17

(52) U.S. Cl. CPC G01T 1/17
USPC 250/394; 250/336.1; 250/395

(57) ABSTRACT

A radiation irradiation initiation detection apparatus includes an acquisition unit, an averaging unit, a calculation unit and a determination unit. The acquisition unit acquires a detection result for each of frames from a detection section that detects radiation. The averaging unit averages the detection results of a plural number of frames, which detection results have been previously acquired by the acquisition unit. The calculation unit calculates at least one of a difference or a ratio between the most recent detection result acquired by the acquisition unit and an averaging result from the averaging unit. The determination unit determines whether or not irradiation of radiation has been initiated on the basis of calculation results from the calculation unit.

### CROSS-REFERENCE TO RELATED APPLICATIONS

This application claims priority under 35 USC 119 from Japanese Patent Application No. 2012-016682 filed on Jan. 30, 2012, the disclosure of which is incorporated by reference herein.

### BACKGROUND OF THE INVENTION

1. Field of the Invention

The present invention relates to a radiation irradiation initiation determination apparatus, a radiation image capturing device, a radiation image capture control apparatus, a radiation irradiation initiation determination method, and a computer readable medium.

2. Description of the Related Art

In recent years, radiation detectors such as flat panel detectors (FPD) and the like have been realized. In an FPD, a radiation-sensitive layer is disposed on a thin film transistor (TFT) active matrix substrate, and the FPD is capable of converting radiation directly to digital data. A radiation image capture device that uses this radiation detector to capture radiation images expressed by irradiated radiation has been realized. A system for converting radiation in the radiation detector used in this radiation image capturing device is of: an indirect conversion type that converts radiation to light with a scintillator and then converts the converted light to electronic charges in a semiconductor layer of photodiodes or the like; a direct conversion type that converts radiation to electronic charges in a semiconductor layer of amorphous selenium or the like; or the like. Whatever the system, there are a variety of materials that may be used in the semiconductor layer.

As this kind of radiation image capturing device, Japanese Patent Application Laid-Open (JP-A) No. 2011-193306 proposes a radiation image capturing device capable of detecting the initiation of irradiation of radiation.

In the technology recited in Japanese Patent Application Laid-Open (JP-A) No. 2011-193306, in a case in which a period of reading out data from all radiation detection elements of a detection section is a single frame, a controller repeatedly performs, for each frame: image data readout processing that applies an On voltage to a signal line and reads out image data from the radiation detection elements connected to that signal line; and leak data readout processing that, in a state in which the On voltage is not applied to the signal line, reads out a total value of electronic charges leaking from the radiation detection elements to be used as leak data for the respective signal line. The controller detects the initiation of irradiation of radiation on the basis of the image data read out by the readout processing. In each of a predetermined number of frames including a frame for which the image data readout processing has been performed at the moment at which the irradiation of radiation initiated, the controller acquires the image data and leak data for each frame and for each radiation detection element. In the technology of JP-A No. 2011-193306, it is recited that a value that is a predetermined value added to an average value of image data for a number of frames serves as a threshold value for detecting the initiation of irradiation of radiation.

In JP-A No. 2007-75598, in order to reduce an offset component and random noise or the like, it is proposed to subtract a signal value for correction, which is obtained from signal values read out before and after irradiation of radiation, from signal values read out during the irradiation of radiation.

However, with the technology recited in JP-A No. 2011-193306, it may be mistakenly judged that irradiation of radiation has been initiated in a case in which there is a defective radiation detection component that outputs substantial image data even when radiation is not being irradiated, a case in which delays with unexpectedly large values occur in the image data, or the like. It is judged that irradiation of radiation has been initiated if an individual value of read-out image data, the accumulated value of image data for a respective line of the signal lines, or a sum of image data of a respective frame exceeds a threshold value. Thus, because detection signals of a single frame are used, the initiation of irradiation of radiation is mistakenly detected in a case in which there is an abnormality for a single frame. Therefore, there is room for improvement.

In JP-A No. 2007-75598, signals from before and after the irradiation of radiation are required in order to correct the offset component, random noise and the like. The technology recited in JP-A No. 2007-75598 may not be used for noise removal during detection for the initiation of irradiation of radiation.

In the technology recited in JP-A No. 2011-193306, it is recited that the initiation of irradiation of radiation is detected with the threshold value being a value for which the predetermined value is added to the average value of image data of several frames. For a number of frames in an initial period, in which dark currents are large, the initiation of irradiation may be detected from the dark currents even though irradiation of radiation has not initiated. Therefore, the initiation of irradiation of radiation may not be detected from the first several frames, and time is needed before the initiation of irradiation of radiation can be detected.

### SUMMARY OF THE INVENTION

The present invention has been made in view of the above circumstances and provides a radiation irradiation initiation determination apparatus, a radiation image capturing device, a radiation image capture control apparatus, a radiation irradiation initiation determination method, and a computer readable medium.

According to an aspect of the present invention, there is provided a radiation irradiation initiation determination apparatus including: an acquisition unit that acquires a detection result for each of frames from a detection section that detects radiation; an averaging unit that averages detection results of a plurality of frames which have been previously acquired by the acquisition unit; a calculation unit that calculates at least one of a difference or a ratio between a most recent detection result acquired by the acquisition unit and an averaging result from the averaging unit; and a determination unit that determines whether or not irradiation of radiation has been initiated, on the basis of a calculation result from the calculation unit.

According to another aspect of the present invention, there is provided a radiation image capturing device including: the radiation irradiation initiation determination apparatus.

According to another aspect of the present invention, there is provided a radiation image capture control apparatus including: the radiation irradiation initiation determination apparatus.

According to another aspect of the present invention, there is provided a radiation irradiation initiation determination method including: acquiring a detection result for each of frames from a detection section that detects radiation; averaging the previously acquired detection results of a plurality of frames; calculating at least one of a difference or a ratio between a most recent detection result and a result of the averaging; and determining whether or not irradiation of radiation has been initiated on the basis of a result of the calculating.

According to another aspect of the present invention, there is provided a non-transitory computer readable medium storing a program causing a computer to execute radiation irradiation initiation determination processing, the processing including: acquiring a detection result for each of frames from a detection section that detects radiation; averaging the previously acquired detection results of a plurality of frames; calculating at least one of a difference or a ratio between a most recent detection result and a result of the averaging; and determining whether or not irradiation of radiation has been initiated on the basis of a result of the calculating.

### BRIEF DESCRIPTION OF THE DRAWINGS

Preferred embodiments of the present invention will be described in detail based on the following figures, wherein:

FIG. 1 is a block diagram illustrating the structure of a radiology information system in accordance with an exemplary embodiment.

FIG. 2 is a side elevation showing an example of a state of arrangement of devices in a radiography imaging room of a radiographic image capturing system in accordance with the exemplary embodiment.

FIG. 3 is a sectional schematic diagram showing schematic structure of a three-pixel portion of a radiation detector in accordance with the exemplary embodiment.

FIG. 4 is a sectional side elevation schematically showing the structure of a signal output section of a one-pixel portion of the radiation detector in accordance with the exemplary embodiment.

FIG. 5 is a plan view showing structure of the radiation detector in accordance with the exemplary embodiment.

FIG. 6 is a block diagram showing the structure of principal elements of an electronic system of an imaging system in accordance with the exemplary embodiment.

FIG. 7 is a circuit diagram showing the structure of a second signal processing section in accordance with the exemplary embodiment.

FIG. 8 is a functional block diagram showing the structure of principal elements of a radiation detection determination function of a cassette control section in accordance with the exemplary embodiment.

FIG. 9 is a flowchart showing the flow of processing of a radiation image capture processing program in accordance with the exemplary embodiment.

FIG. 10 is a schematic diagram showing an example of an initial information input screen in accordance with the exemplary embodiment.

FIG. 11 is a flowchart showing the flow of processing of a cassette imaging processing program in accordance with the exemplary embodiment.

FIG. 12 is a diagram for explaining an example of threshold value setting processing.

FIG. 13 is a sectional side elevation for explaining penetration side sampling and irradiation side sampling of radiation images.

FIG. 14 is a diagram showing another structural example of radiation detection pixels.

### DETAILED DESCRIPTION OF THE INVENTION

Herebelow, modes for carrying out the present invention are described in detail with reference to the attached drawings. Herein, an example of a case in which the present invention is applied to a radiology information system, which is a system that collectively administers information managed by a radiology department in a hospital, is described.

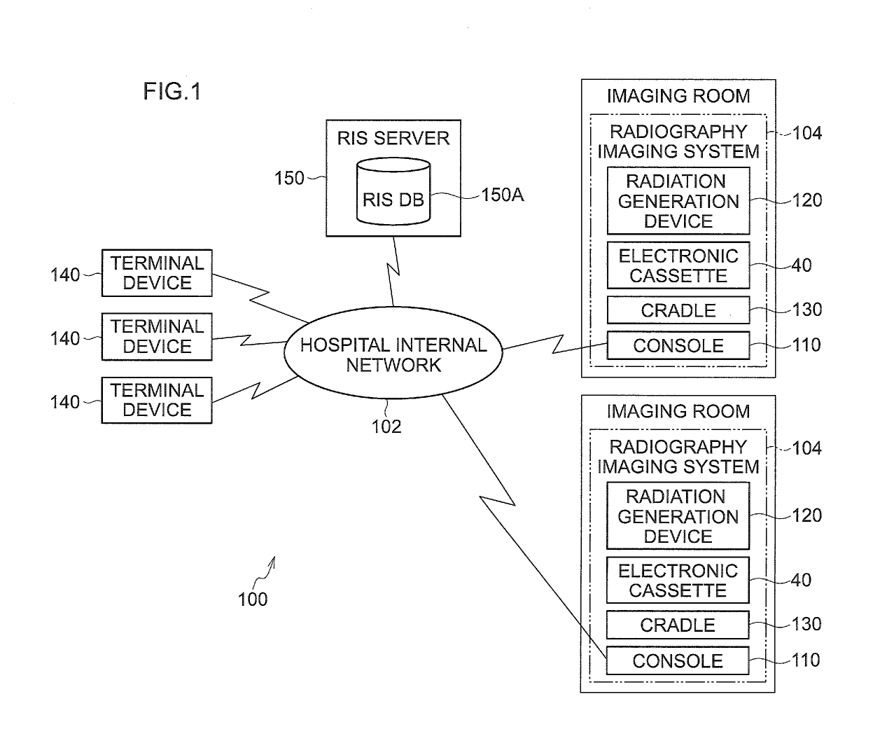

First, a configuration of a radiology information system (hereinafter referred to as an RIS) 100 relating to the present exemplary embodiment is described with reference to FIG. 1.

The RIS 100 is a system for administering information of clinical appointments, medical records and so forth in a radiology department, and constitutes a portion of a hospital information system (hereinafter referred to as an HIS).

The RIS 100 is constituted with a plural number of imaging request terminal devices (hereinafter referred to as terminal devices) 140, an RIS server 150 and a radiographic image capture system (hereinafter referred to as an imaging system) 104, which is separately installed in a radiography imaging room (or an operating room) in the hospital, being connected to a hospital internal network 102, which is formed with a wired or wireless local area network (LAN) or the like. Herein, the RIS 100 constitutes a portion of the HIS provided in the same hospital, and an HIS server (not shown in the drawings) that administers the HIS as a whole is also connected to the hospital internal network 102.

Each terminal device 140 is for a doctor, a radiographer or the like to input and monitor clinical information, facility reservations and the like. Imaging requests for radiographic images, imaging bookings and the like are also conducted through the terminal device 140. The terminal device 140 includes a personal computer with a display device, and is connected with the RIS server 150 via the hospital internal network 102, enabling communications therebetween.

The RIS server 150 receives imaging requests from the terminal devices 140 and manages an imaging schedule for radiographic images at the imaging system 104. The RIS server 150 includes a database 150A.

The database 150A is constituted to include: information relating to patients, such as information on attributes (name, gender, date of birth, age, blood type, body weight, a patient identification (ID) number and so forth) of each patient (imaging subject), medical record, treatment history, previously imaged radiographic images, and the like; information relating to electronic cassettes 40 of the imaging system 104 which are described below, such as an identification number (ID information) of each electronic cassette 40 and the type, size, sensitivity, the date of first use, the number of uses, and the like; and environmental information representing environments in which the electronic cassettes 40 are used to capture radiographic images, which is to say environments in which the electronic cassettes 40 are employed (for example, a radiographic imaging room, an operating room and the like).

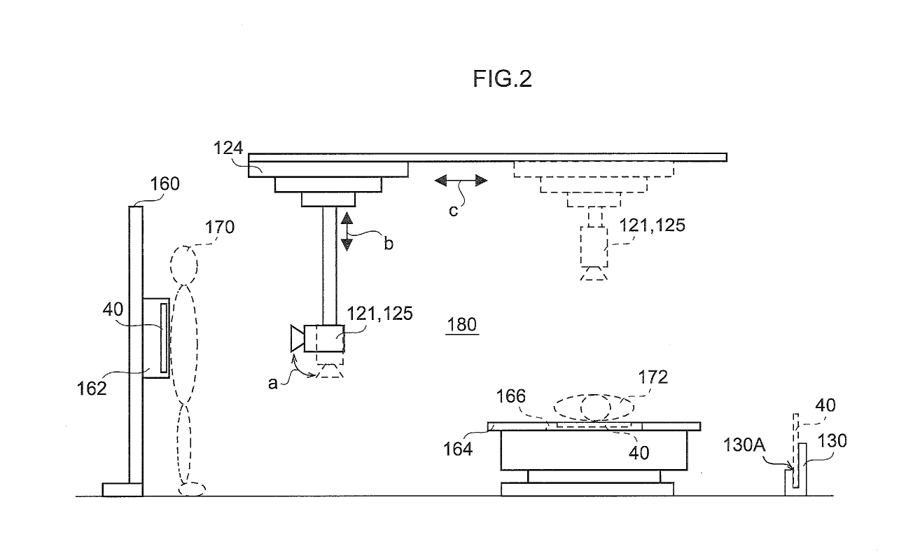

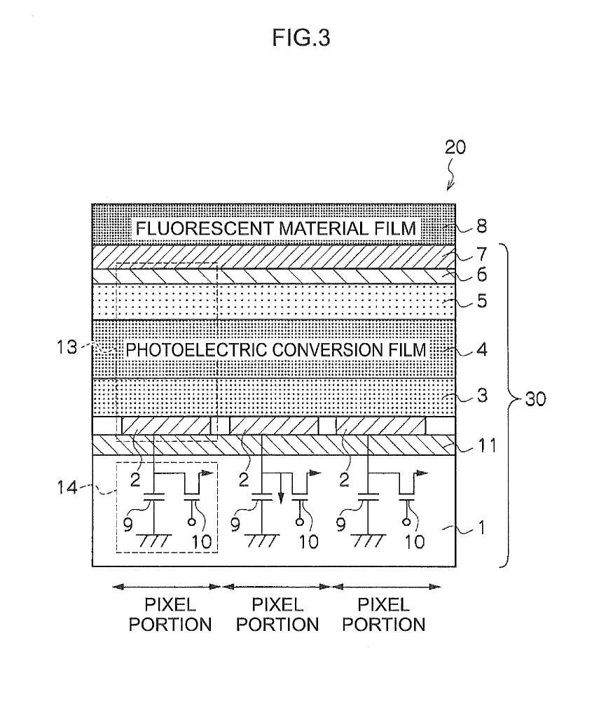

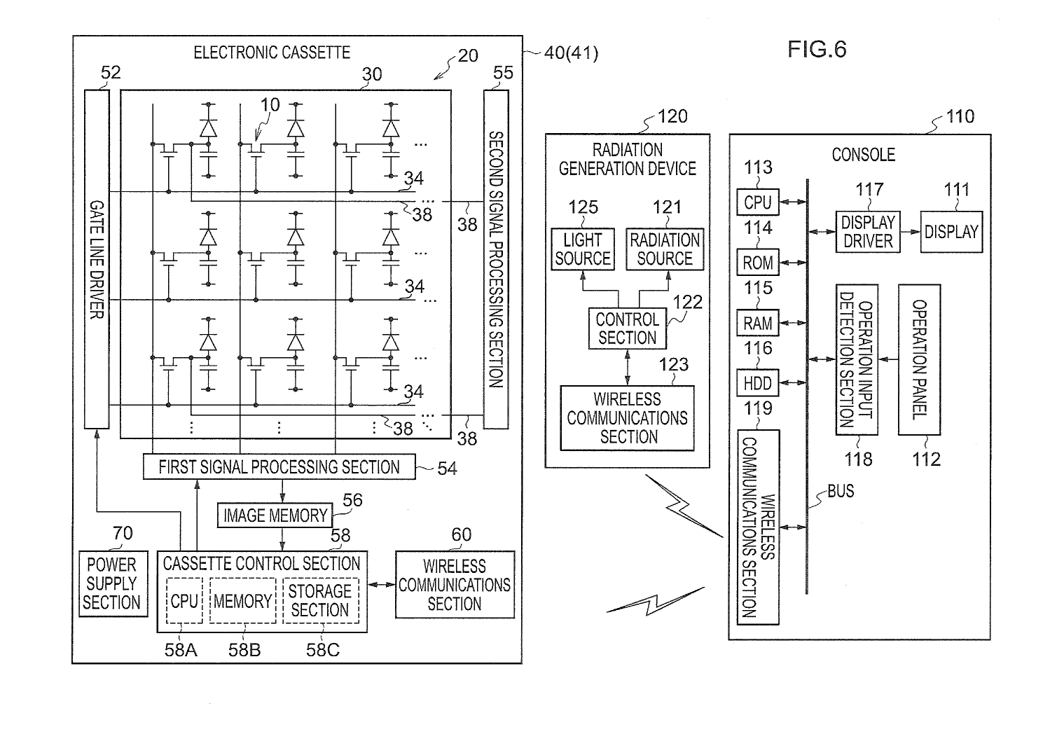

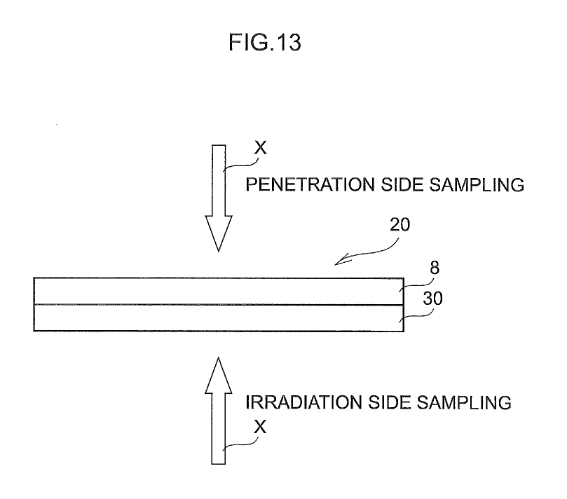

The imaging system 104 carries out imaging of radiographic images in response to instructions from the RIS server 150, in accordance with control by doctors, radiographers and the like. The imaging system 104 is provided with a radiation generation device 120, which irradiates radiation X (see FIG. 13), constituted with radiation amounts depending on exposure conditions, from a radiation source 121 at an imaging subject (see FIG. 2) and, before irradiating the radiation X at the imaging subject, illuminates visible light from a light source 125 for positioning of the imaging subject with respect to irradiation field of the radiation X (see FIG. 2). The imaging system 104 is also provided with the electronic cassette 40, which incorporates a radiation detector 20, a cradle 130, which charges a battery incorporated in the electronic cassette 40, and a console 110, which controls the electronic cassette 40 and the radiation generation device 120. The radiation detector 20 absorbs the radiation X that has passed through an imaging target portion of an imaging subject and generates electronic charges and, on the basis of the generated charge amounts, generates image information representing a radiographic image (see FIG. 3 and FIG. 6).

The console 110 acquires various kinds of information contained in the database 150A from the RIS server 150, stores the information in a hard disc drive (HDD) 116 (see FIG. 6), which is described below, and controls the electronic cassette 40 and the radiation generation device 120 using this information in accordance with needs.

FIG. 2 illustrates an example of a state of arrangement of devices in a radiography imaging room 180 of the imaging system 104 according to the present exemplary embodiment.

As shown in FIG. 2, in the radiography imaging room 180, a standing position stand 160 that is used when radiographic imaging is being carried out on an imaging subject in a standing position and a lying position table 164 that is used when radiographic imaging is being carried out on an imaging subject in a lying position are provided. A space forward of the standing position stand 160 serves as an imaging position 170 of the imaging subject when radiographic imaging is being carried out in the standing position, and a space upward of the lying position table 164 serves as an imaging position 172 of the imaging subject when radiographic imaging is being carried out in the lying position.

A retention portion 162 that retains the electronic cassette 40 is provided at the standing position stand 160. When a radiographic image is being imaged in the standing position, the electronic cassette 40 is retained by the retention portion 162. Similarly, a retention portion 166 that retains the electronic cassette 40 is provided at the lying position table 164. When a radiographic image is being imaged in the lying position, the electronic cassette 40 is retained by the retention portion 166.

In the radiography imaging room 180, in order that both radiographic imaging in the standing position and radiographic imaging in the lying position are possible with radiation from the single radiation source 121, a support and movement mechanism 124 is provided that supports the radiation source 121 and the light source 125 to be turnable (in the direction of arrow a in FIG. 2) about a horizontal axis, movable in a vertical direction (the direction of arrow b in FIG. 2) and movable in a horizontal direction (the direction of arrow c in FIG. 2). The support and movement mechanism 124 is provided with each of a drive source that turns the radiation source 121 and light source 125 about the horizontal axis, a drive source that moves the radiation source 121 and light source 125 in the vertical direction, and a drive source that moves the radiation source 121 and light source 125 in the horizontal direction (none of which are shown in the drawings).

In the cradle 130, an accommodation portion 130A capable of accommodating the electronic cassette 40 is formed.

When the electronic cassette 40 is accommodated in the accommodation portion 130A of the cradle 130, the battery incorporated in the electronic cassette 40 is charged up. When a radiographic image is to be imaged, the electronic cassette 40 is taken from the cradle 130 by a radiographer or the like. If a posture for imaging is to be the standing position, the electronic cassette 40 is retained at the retention portion 162 of the standing position stand 160, and if the posture for imaging is to be the lying position, the electronic cassette 40 is retained at the retention portion 166 of the lying position table 164.

In the imaging system 104 according to the present exemplary embodiment, various kinds of information are exchanged by wireless communications between the radiation generation device 120 and the console 110 and between the electronic cassette 40 and the console 110.

The electronic cassette 40 is not used only in conditions in which it is retained by the retention portion 162 of the standing position stand 160 or the retention portion 166 of the lying position table 164. The electronic cassette 40 is portable, and therefore may be used in conditions in which it is not retained at a retention portion, for imaging arm areas, leg areas or the like.

Next, structure of the radiation detector 20 relating to the present exemplary embodiment is described. FIG. 3 is a sectional schematic diagram schematically showing the structure of three-pixel portions of the radiation detector 20 according to the present exemplary embodiment. In the present exemplary embodiment, an example is described in which an indirect conversion type of the radiation detector 20 is employed. However, a direct conversion-type radiation detector may be employed.

As shown in FIG. 3, in the radiation detector 20 according to the present exemplary embodiment, signal output sections 14, sensor sections 13 and a scintillator 8 are sequentially layered on an insulating substrate 1, and pixels are constituted by the signal output sections 14 and sensor sections 13. The pixels are plurally arrayed on the substrate 1 and, at each pixel, the signal output section 14 and sensor section 13 are superposed.

The scintillator 8 is formed over the sensor sections 13 with a transparent insulating film 7 therebetween. The scintillator 8 is a film formed of a fluorescent material that converts radiation that is incident from above (the opposite side thereof from the side at which the substrate 1 is disposed) or below to light and emits the light. Because of the provision of the scintillator 8, radiation that has passed through an imaging subject is absorbed and light is emitted.

The wavelength range of the light emitted by the scintillator 8 is preferably in the visible light range (wavelengths from 360 nm to 830 nm). To enable monochrome imaging by the radiation detector 20, it is more preferable if a green wavelength range is included.

Each sensor section 13 includes an upper electrode 6, a lower electrode 2, and a photoelectric conversion film 4 disposed between the upper and lower electrodes. The photoelectric conversion film 4 is constituted with an organic photoelectric conversion material that absorbs the light emitted by the scintillator 8 and generates charges.

The photoelectric conversion film 4 includes an organic photoelectric conversion material, absorbs light emitted from the scintillator 8, and generates electric charges in accordance with the absorbed light. If the photoelectric conversion film 4 includes an organic photoelectric conversion material, the film has a sharp absorption spectrum in the visible range and hardly any electromagnetic waves apart from the light emitted by the scintillator 8 are absorbed by the photoelectric conversion film 4. Thus, noise due to the absorption of radiation such as X-rays and the like at the photoelectric conversion film 4 may be effectively suppressed.

It is sufficient if the sensor section 13 constituting each pixel includes at least the lower electrode 2, the photoelectric conversion film 4 and the upper electrode 6. However, to restrain an increase in dark currents, it is preferable to provide at least one of an electron blocking film 3 and a hole blocking film 5, and it is more preferable to provide both.

The electron blocking film 3 may be provided between the lower electrodes 2 and the photoelectric conversion film 4. If a bias voltage is applied between the lower electrodes 2 and the upper electrode 6, electrons are injected from the lower electrodes 2 into the photoelectric conversion film 4 and an increase in dark currents may be suppressed.

The hole blocking film 5 may be provided between the photoelectric conversion film 4 and the upper electrode 6. If a bias voltage is applied between the lower electrodes 2 and the upper electrode 6, holes are injected from the upper electrode 6 into the photoelectric conversion film 4 and an increase in dark currents may be suppressed.

If a bias voltage is specified such that, of the charges produced in the photoelectric conversion film 4, holes migrate to the upper electrode 6 and electrons migrate to the lower electrodes 2, the positions of the electron blocking film 3 and the hole blocking film 5 may be exchanged. It may be that neither the electron blocking film 3 nor the hole blocking film 5 is provided, but if either is provided, a dark current suppression effect may be obtained to some extent.

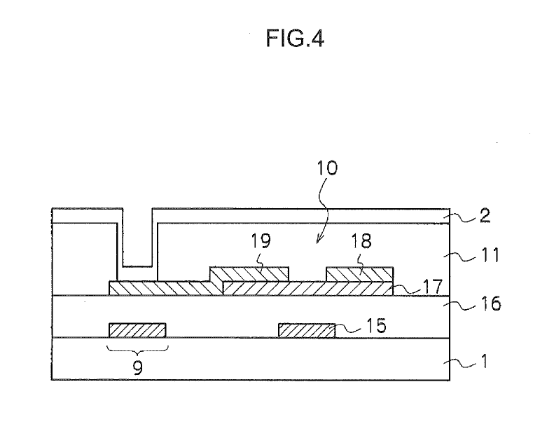

Each signal output section 14 is formed on the surface of the substrate 1 below the lower electrode 2 of the pixel. The structure of the signal output section 14 is schematically illustrated in FIG. 4.

As shown in FIG. 4, each signal output section 14 according to the present exemplary embodiment is formed with a capacitor 9, which corresponds with the lower electrode 2 and accumulates charges that have migrated to the lower electrode 2, and a field effect-type thin film transistor (hereinafter referred to simply as a thin film transistor) 10, which converts the charges accumulated at the capacitor 9 to electronic signals and outputs the electronic signals. A region in which the capacitor 9 and thin film transistor 10 are formed includes a region that overlaps with the lower electrode 2 in plan view. Because of this structure, the signal output section 14 and the sensor section 13 are superposed in the thickness direction. To minimize a planar area of the radiation detector 20 (the pixels), it is desirable if the region in which each capacitor 9 and thin film transistor 10 is formed is completely covered by the lower electrode 2.

An insulating film 11 is provided between the substrate 1 and the lower electrode 2. The capacitor 9 is electrically connected with the corresponding lower electrode 2 via wiring of a conductive material that is formed to penetrate through the insulating film 11. Thus, charges collected at the lower electrode 2 may be allowed to migrate to the capacitor 9.

In each thin film transistor 10, a gate electrode 15, a gate insulation film 16 and an active layer (a channel layer) 17 are layered. A source electrode 18 and a drain electrode 19 are formed, with a predetermined gap formed therebetween, on the active layer 17.

In the present exemplary embodiment, a TFT substrate 30 is formed on the substrate 1 by sequential formation of the signal output sections 14, the sensor sections 13 and the transparent insulating film 7. The radiation detector 20 is formed by the scintillator 8 being adhered onto the TFT substrate 30 using an adhesive resin or the like with low light absorption.

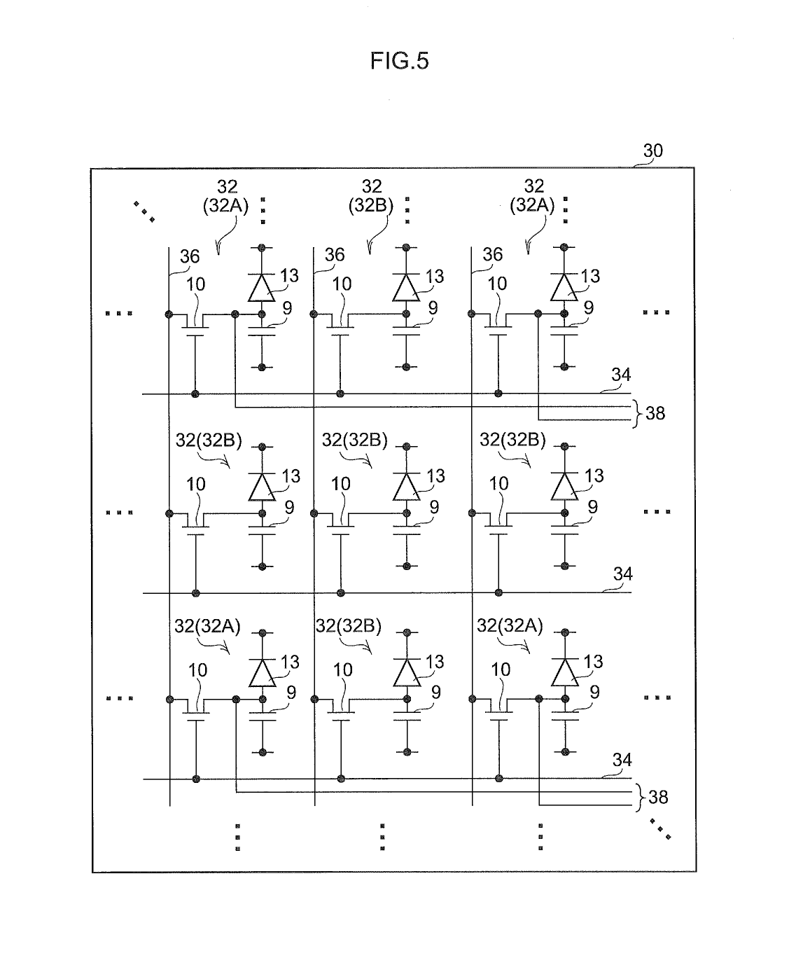

As shown in FIG. 5, pixels 32 are plurally provided in two dimensions on the TFT substrate 30, in a certain direction (a scan line direction in FIG. 5, which is hereinafter referred to as the row direction), and a direction orthogonal to the certain direction (a signal line direction in FIG. 5, which is hereinafter referred to as the column direction). Each pixel 32 is constituted to include the above-described sensor section 13, capacitor 9 and thin film transistor 10.

Plural gate lines 34 and plural data lines 36 are provided in the radiation detector 20. The gate lines 34 extend in the certain direction (the row direction) and are for turning the thin film transistors 10 on and off. The data lines 36 extend in the direction orthogonal to the gate lines 34 (the column direction) and are for reading out the charges via the thin film transistors 10 that have been turned on.

The radiation detector 20 has a flat-plate form, and is formed in a quadrilateral shape with four outer edges in plan view, and more specifically a rectangular shape.

In the radiation detector 20 according to the present exemplary embodiment, some of the pixels 32 are used for detecting radiation irradiation states, and a radiation image is captured by the rest of the pixels 32. Hereinafter, the pixels 32 for detecting radiation irradiation states are referred to as radiation detection pixels 32A, and the other pixels 32 are referred to as radiation image acquisition pixels 32B.

In the radiation detector 20 according to the present exemplary embodiment, because a radiation image is captured by the radiation image acquisition pixels 32B of the pixels 32 excluding the radiation detection pixels 32A, pixel information of the radiation image may not be acquired for the positions at which the radiation detection pixels 32A are disposed. Accordingly, the radiation detection pixels 32A are disposed so as to be scattered in the radiation detector 20 according to the present exemplary embodiment, and missing pixel correction processing is executed by the console 110, which generates pixel information of the radiation image for each position, at which the radiation detection pixels 32A are disposed, by interpolation using image information acquired by the radiation image acquisition pixels 32B disposed around that radiation detection pixel 32A.

Furthermore, in the radiation detector 20 according to the present exemplary embodiment, the radiation detection pixels 32A are disposed in the imaging region so as to have a higher density in regions at which an imaging target portion is not disposed and which are more frequently absent regions (through regions).

To detect radiation irradiation states, the electronic cassette 40 according to the present exemplary embodiment is provided with a radiation amount acquisition function that acquires information representing irradiation amounts of the radiation X from the radiation source 121 (hereinafter referred to as radiation amount information).

Accordingly, in the radiation detector 20 according to the present exemplary embodiment, as shown in FIG. 5, direct connection readout wires 38 are separately provided extending in the certain direction (the row direction) from each of the radiation detection pixels 32A. Each direct connection readout wire 38 is connected with a section connecting between the capacitor 9 and the thin film transistor 10 in the radiation detection pixel 32A, and is for directly reading out charges accumulated in the capacitor 9.

Next, the structure of principal portions of an electrical system of the imaging system 104 according to the present exemplary embodiment is described with reference to FIG. 6.

As shown in FIG. 6, the radiation detector 20 incorporated in the electronic cassette 40 is provided with a gate line driver 52, which is disposed at one of two adjoining sides of the radiation detector 20, and a first signal processing section 54, which is disposed at the other of the two adjoining sides. The individual gate lines 34 of the TFT substrate 30 are connected to the gate line driver 52, and the individual data lines 36 of the TFT substrate 30 are connected to the first signal processing section 54.

An image memory 56, a cassette control section 58 and a wireless communications section 60 are also provided inside a casing 41.

The thin film transistors 10 of the TFT substrate 30 are sequentially turned on in row units by signals provided from the gate line driver 52 via the gate lines 34, and charges that are read out by the thin film transistors 10 that have been turned on are propagated through the data lines 36 as electronic signals and inputted to the first signal processing section 54. Thus, the charges are sequentially read out row by row, and a two-dimensional radiation image may be acquired.

Although not shown in the drawings, the first signal processing section 54 is provided with an amplification circuit and a sample and hold circuit for each of the data lines 36. The amplification circuits amplify the inputted electronic signals. After the electronic signals that have been propagated through the respective data lines 36 are amplified by the amplification circuits, the amplified signals are retained at the sample and hold circuits. At the output side of the sample and hold circuits, a multiplexer and an analog-to-digital (A/D) converter are connected in this order. The electronic signals retained at the respective sample and hold circuits are sequentially (serially) inputted to the multiplexer, and are converted to digital image data by the A/D converter.

The image memory 56 is connected to the first signal processing section 54, and the image data outputted from the A/D converters of the first signal processing section 54 is sequentially stored in the image memory 56. The image memory 56 has a storage capacity capable of storing a predetermined number of frames of image data. Each time a radiographic image is captured, image data obtained by the imaging is sequentially stored in the image memory 56.

The image memory 56 is connected to the cassette control section 58. The cassette control section 58 includes a microcomputer, and is provided with a central processing unit (CPU) 58A, a memory 58B including a read-only memory (ROM) and random access memory (RAM), and a non-volatile storage section 58C formed of flash memory or the like. The cassette control section 58 controls overall operations of the electronic cassette 40.

The wireless communications section 60 is connected to the cassette control section 58. The wireless communications section 60 complies with wireless LAN (local area network) standards, typified by IEEE (Institute of Electrical and Electronics Engineers) standards 802.11 a/b/g and the like. The wireless communications section 60 controls transfers of various kinds of information between the cassette control section 58 and an external equipment by wireless communications. The cassette control section 58 is capable of wireless communications, via the wireless communications section 60, with external devices such as the console 110 that controls the capture of radiation images and the like, and may exchange various kinds of information with the console 110 and the like.

The electronic cassette 40 is also provided with a power supply section 70. The various circuits and elements mentioned above (the gate line driver 52, the first signal processing section 54, the image memory 56, the wireless communications section 60, the microcomputer that functions as the cassette control section 58, and the like) are driven by electrical power supplied from the power supply section 70. The power supply section 70 incorporates a battery (a rechargeable secondary cell), so as not to impede portability of the electronic cassette 40, and provides power to the various circuits and elements from the charged battery. Wiring connecting the power supply section 70 with the various circuits and elements is not shown in FIG. 6.

The radiation detector 20 according to the present exemplary embodiment is also provided with a second signal processing section 55 for implementing the above-mentioned radiation amount acquisition function, at the opposite side of the TFT substrate 30 from the side thereof at which the gate line driver 52 is disposed. The individual direct connection readout wires 38 of the TFT substrate 30 are connected to the second signal processing section 55.

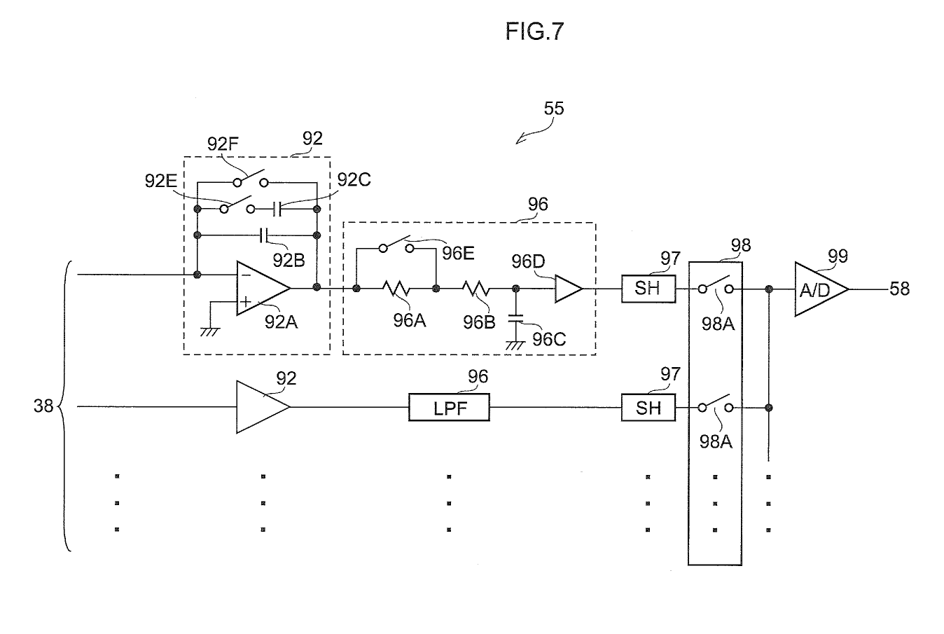

Next, the structure of the second signal processing section 55 relating to the present exemplary embodiment is described. FIG. 7 shows a circuit diagram illustrating the structure of the second signal processing section 55 according to the present exemplary embodiment.

As shown in FIG. 7, for each of the direct connection readout wires 38, the second signal processing section 55 according to the present exemplary embodiment is provided with a variable gain preamplifier (charge amplifier) 92, a low pass filter (LPF) 96 whose low pass frequency may be switched, and a sample and hold circuit 97 whose sample timing may be set.

The variable gain preamplifier 92 includes an operational amplifier 92A, whose non-inverting input side is connected to ground, and a capacitor 92B, a switch 92E, a capacitor 92C and a reset switch 92F, which are connected between the inverting input side and the output side of the operational amplifier 92A. The capacitor 92B, the switch 92E and capacitor 92C, and the reset switch 92F are connected in parallel with one another. The switch 92E and the reset switch 92F can be switched by the cassette control section 58.

The LPF 96 includes a resistor 96A, a resistor 96B, a capacitor 96C, and a switch 96E that shorts out the resistor 96A. The switch 96E can be switched by the cassette control section 58. The sample timing of the sample and hold circuit 97 can also be switched by the cassette control section 58.

The second signal processing section 55 according to the present exemplary embodiment is also provided with a single multiplexer 98 and a single analog-to-digital (A/D) converter 99. Output selection can be switched by the cassette control section 58 using switches 98A provided in the multiplexer 98.

Each of the direct connection readout wires 38 is connected to the input terminal of the corresponding variable gain preamplifier 92 (i.e., the inverting input side of the operational amplifier 92A). The output terminal of the variable gain preamplifier 92 is connected to the input terminal of the corresponding LPF 96, and the output terminal of the LPF 96 is connected to the input terminal of the corresponding sample and hold circuit 97.

The respective output terminals of the sample and hold circuits 97 are connected to the switches 98A of the multiplexer 98 in a one-to-one correspondence, and output terminals of the switches 98A of the multiplexer 98 are connected to an input terminal of the A/D converter 99, which is connected to the cassette control section 58.

When the radiation amount acquisition function is operated, the cassette control section 58 first discharges charges that have accumulated at the capacitor 92B and capacitor 92C of each variable gain preamplifier 92 by turning on the switch 92E and reset switch 92F.

Then, the cassette control section 58 sets the amplification ratio of the variable gain preamplifier 92 by setting the reset switch 92F of the variable gain preamplifier 92 to off and setting the switch 92E to on or off. The cassette control section 58 also sets the low pass frequency of the LPF 96 by setting the switch 96E of the LPF 96 to on or off.

Charges that are accumulated at the capacitor 9 of each of the radiation detection pixels 32A due to the radiation X being irradiated are propagated through the direct connection readout wires 38 connected thereto in the form of electronic signals. The electronic signals propagated through the direct connection readout wires 38 are each amplified by the variable gain preamplifier 92 with the amplification ratio set by the cassette control section 58, and then subjected to filtering processing by the LPF 96 at the low pass frequency set by the cassette control section 58.

After the above-described setting of the amplification ratio and the low pass frequency, the cassette control section 58 retains a signal level of the electronic signals that have been subjected to the filtering processing at the sample and hold circuit 97, by driving the sample and hold circuit 97 for a predetermined period.

The signal levels retained at the sample and hold circuits 97 are sequentially selected by the multiplexer 98 in accordance with control by the cassette control section 58, and are A/D converted by the A/D converter 99. Then, the digital data that is obtained is outputted to the cassette control section 58. The digital data outputted from the A/D converter 99 represents radiation amounts irradiated onto the radiation detection pixels 32A in the predetermined duration, and is used for creating the aforementioned radiation amount information.

At the cassette control section 58, the digital data corresponding to the respective radiation detection pixels 32A that is inputted from the A/D converter 99 is stored in a pre-specified region of the RAM of the memory 58B.

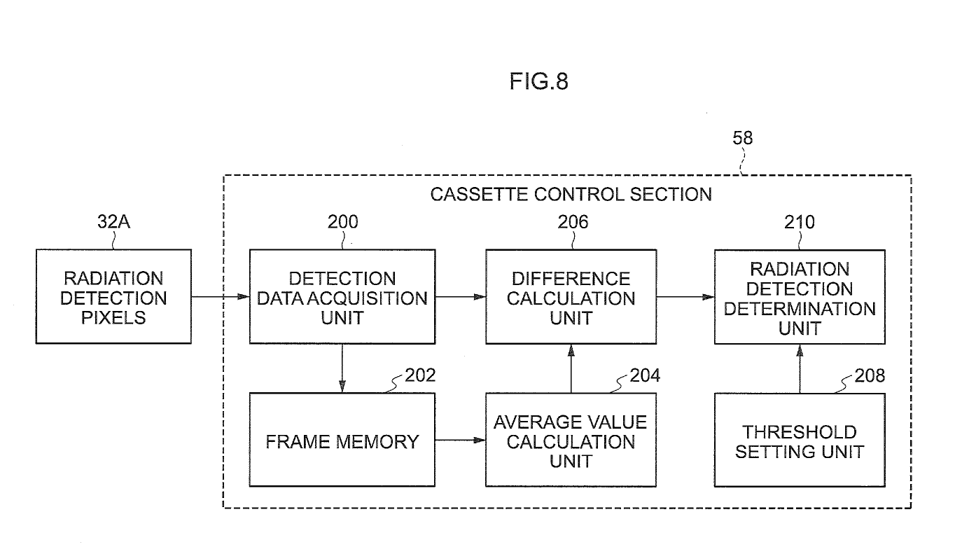

The cassette control section 58 includes a radiation detection determination function that determines whether or not irradiation of the radiation has been initiated on the basis of the radiation amount information created by the above-mentioned radiation amount acquisition function. Now, the radiation detection determination function is described. FIG. 8 is a functional block diagram showing schematic structure of the radiation detection determination function of the cassette control section 58 in accordance with an exemplary embodiment of the present invention. The radiation detection determination function illustrated in FIG. 8 may be implemented by hardware structures such as a logic circuit or the like, and may be implemented by software structures such as a program or the like.

As shown in FIG. 8, the cassette control section 58 is provided with the functions of a detection data acquisition unit 200, a frame memory 202, an average value calculation unit 204, a difference calculation unit 206, a threshold setting unit 208 and a radiation detection determination unit 210.

Detection data (digital data) obtained from the radiation detection pixels 32A via the second signal processing section 55 is acquired by the detection data acquisition unit 200, and the acquired detection data is both stored in the frame memory 202 and outputted to the difference calculation unit 206. The second signal processing section 55 is not shown in FIG. 8.

The frame memory 202 is capable of storing detection data corresponding to several frames (in the present exemplary embodiment, four frames), and is sequentially overwritten with the detection data of new frames. The frame memory 202 outputs the stored detection data corresponding to four frames to the average value calculation unit 204.

The detection data of several frames is averaged by calculating an average value of the detection data of the immediately preceding several frames (four frames in the present exemplary embodiment). In other words, the average value calculation unit 204 calculates a moving average of the several frames.

The difference calculation unit 206 calculates a difference between the most recent detection data acquired by the detection data acquisition unit 200 and the detection data average value of the immediately preceding several frames stored in the frame memory 202, which is calculated by the average value calculation unit 204. Thus, dark current correction is implemented.

The threshold setting unit 208 contains pre-specified threshold values corresponding to numbers of frames when the average values are being calculated by the average value calculation unit 204, and sets a threshold value in accordance with a number of object frames for calculating an average value. Specifically, in the present exemplary embodiment, there are four thresholds: a first threshold value for a case in which the number of frames when the average values are calculated by the average value calculation unit 204 is one, a second threshold value for a case of two frames, a third threshold value for a case of three frames, and a fourth threshold value for a case of four frames. In accordance with the number of object frames for the calculation of an average value, the threshold setting unit 208 specifies the corresponding threshold value. The threshold values are set to be smaller when the number of object frames for the calculation of an average is larger: the first threshold value>the second threshold value>the third threshold value>the fourth threshold value. If there have been more than four frames, the threshold value is fixed at the fourth threshold value. Herein, a threshold value for frames before dark currents stabilize (for example, for a number of frames in an initial period) may be set to a larger value than a threshold value for when the dark currents are stable.

The radiation detection determination unit 210 identifies irradiation of radiation by determining whether or not a result of calculation by the difference calculation unit 206 exceeds a threshold value set by the threshold setting unit 208. That is, it is judged that radiation has been irradiated in a case in which the calculation result of the difference calculation unit 206 exceeds the threshold value set by the threshold setting unit 208.

As shown in FIG. 6, the console 110 is structured as a server computer. The console 110 is provided with a display 111, which displays control menus, captured radiographic images and the like, and an operation panel 112, which is structured to include plural buttons and at which various kinds of information and control instructions can be inputted.

The console 110 relating to the present exemplary embodiment is provided with: a CPU 113 that administers operations of the device as a whole; a ROM 114 at which various programs, including a control program, and suchlike are stored in advance; a RAM 115 that temporarily stores various kinds of data; the HDD 116, which stores and retains various kinds of data; a display driver 117 that controls displays of various kinds of information at the display 111; and an operation input detection section 118 that detects control states of the operation panel 112. The console 110 is further provided with a wireless communications section 119 that, by wireless communications, exchanges various kinds of information such as the aforementioned exposure conditions and the like with the radiation generation device 120 and exchanges various kinds of information such as image data and the like with the electronic cassette 40.

The CPU 113, ROM 114, RAM 115, HDD 116, display driver 117, operation input detection section 118 and wireless communications section 119 are connected to one another by a system bus. Thus, the CPU 113 may access the ROM 114, RAM 115 and HDD 116, control displays of various kinds of information at the display 111 via the display driver 117 and, via the wireless communications section 119, control transmission and reception of various kinds of information to and from the radiation generation device 120 and the electronic cassette 40. The CPU 113 may also acquire states of operation by users from the operation panel 112 via the operation input detection section 118.

The radiation generation device 120 is provided with the radiation source 121, the light source 125, a wireless communications section 123, and a control section 122. The wireless communications section 123 exchanges various kinds of information such as the exposure conditions and the like with the console 110. The control section 122 controls the radiation source 121 on the basis of received exposure conditions and controls light emission conditions from the light source 125.

The control section 122 is configured to include a microcomputer, and stores the received exposure conditions and the like. The exposure conditions received from the console 110 include information such as a tube voltage, a tube current and the like. The control section 122 causes the radiation X to be irradiated from the radiation source 121 in accordance with the received exposure conditions and, before the irradiation of the radiation X from the radiation source 121, causes visible light to be illuminated for positioning of the imaging subject with respect to the field of irradiation of the radiation X.

Next, operation of the imaging system 104 relating to the present exemplary embodiment is described.

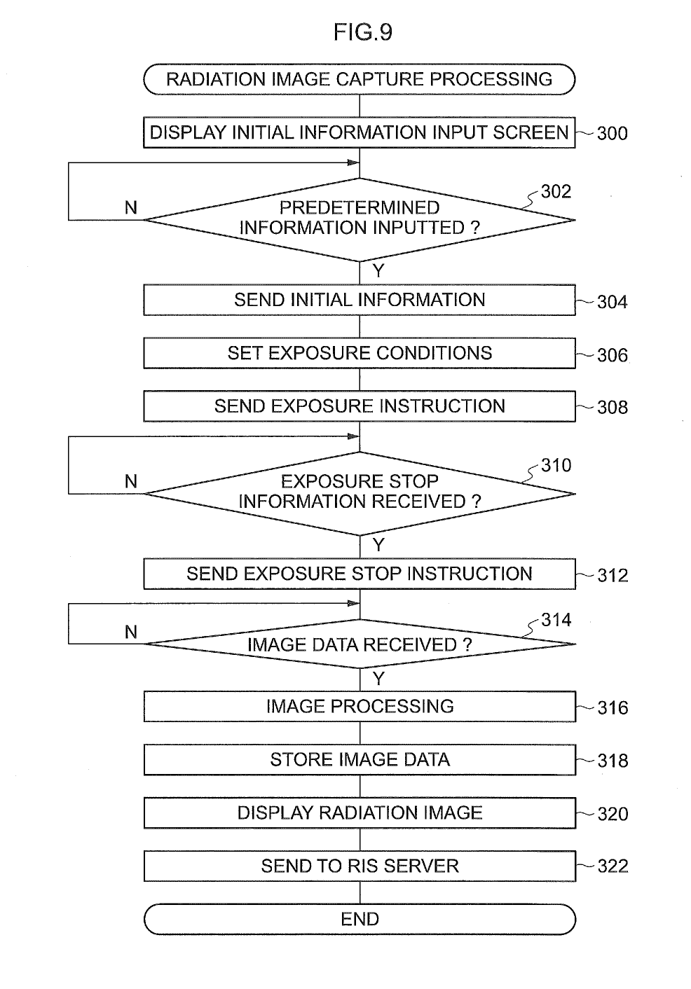

First, operation of the console 110 when capturing a radiographic image is described with reference to FIG. 9. FIG. 9 is a flowchart showing a flow of processing of a radiation image capture processing program that is executed by the CPU 113 of the console 110 when an instruction to execute the same is inputted via the operation panel 112. This program is stored beforehand in a predetermined region of the ROM 114.

In step 300 of FIG. 9, the display driver 117 is controlled such that a pre-specified initial information input screen is displayed by the display 111. Then, in step 302, the CPU 113 waits for the input of predetermined information.

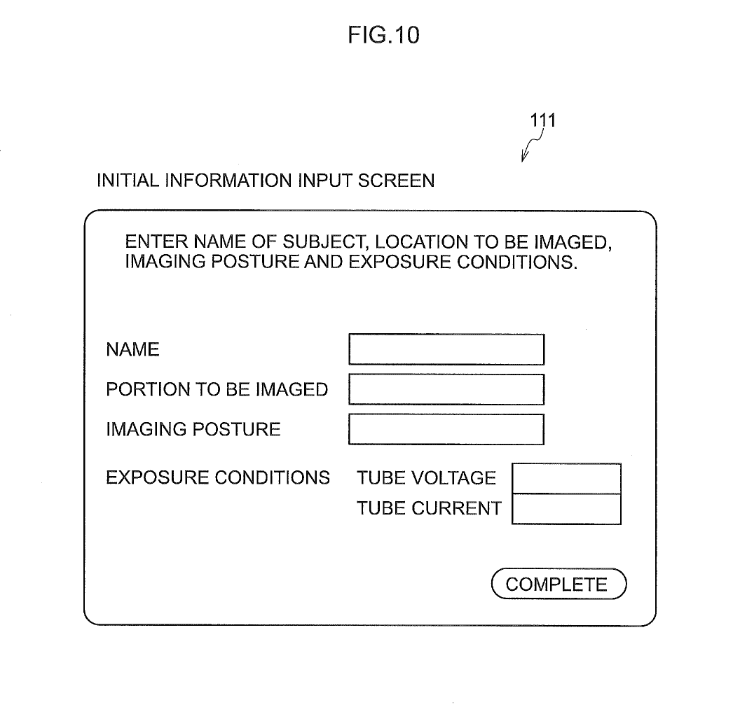

FIG. 10 shows an example of the initial information input screen that is displayed at the display 111 by the processing of step 300. As shown in FIG. 10, the initial information input screen according to the present exemplary embodiment displays a message prompting the input of the name of the subject of whom a radiation image will be captured, the imaging target portion, the subject's posture at the time of imaging, and exposure conditions of the radiation X during the imaging (in the present exemplary embodiment, a tube voltage and tube current when the radiation X is exposed), along with input fields for these items of information.

When the initial information input screen shown in FIG. 10 is displayed at the display 111, the operator inputs at the respectively corresponding input fields, via the operation panel 112, the name of the subject who is the object of imaging, the imaging target portion, the posture at the time of imaging, and the exposure conditions.

Then, the operator enters the radiography imaging room 180 with the imaging subject and, in a case in which the posture during imaging is standing or lying, retains the electronic cassette 40 at the retention portion 162 of the standing position stand 160 or the retention portion 166 of the lying position table 164, positions the electronic cassette 40 at a position that corresponds with the radiation source 121, and then arranges the subject at a predetermined imaging position (positioning). In a case of capturing a radiation image in a state in which the electronic cassette 40 is not retained at a retention portion, when the imaging target portion is an arm area, a leg area or the like, the operator positions the subject, the electronic cassette 40 and the radiation source 121 into a state in which the imaging target portion can be imaged (positioning).

Then, the operator leaves the radiography imaging room 180 and, via the operation panel 112, specifies a Complete button displayed near the bottom end of the initial information input screen. When the Complete button is specified by the operator, the result of the determination in step 302 is affirmative and the CPU 113 proceeds to step 304.

In step 304, the information inputted into the initial information input screen (hereinafter referred to as initial information) is transmitted to the electronic cassette 40 via the wireless communications section 119. Then, in step 306, the exposure conditions included in the initial information are set by transmission of the exposure conditions to the radiation generation device 120 via the wireless communications section 119. Accordingly, the control section 122 of the radiation generation device 120 prepares for exposure with the received exposure conditions.

In step 308, instruction information instructing the initiation of exposure is transmitted to the radiation generation device 120 and the electronic cassette 40 via the wireless communications section 119.

In response, the radiation source 121 initiates emission of the radiation X with the tube voltage and tube current corresponding to the exposure conditions that the radiation generation device 120 received from the console 110. The radiation X emitted from the radiation source 121 reaches the electronic cassette 40 after passing through the imaging subject.

Meanwhile, when the cassette control section 58 of the electronic cassette 40 receives the instruction information instructing the initiation of exposure, the cassette control section 58 creates the radiation amount information using the aforementioned radiation amount acquisition function (described in detail below), and waits until a radiation amount represented by the created radiation amount information is at or above a pre-specified threshold value for detecting that irradiation of radiation has been initiated. Then, the electronic cassette 40 initiates an operation for capturing a radiation image, and subsequently transmits exposure stop information to the console 110 instructing that the exposure of the radiation X be stopped.

Accordingly, in step 310, the console 110 waits for reception of the exposure stop information. Then, in step 312, instruction information instructing that the exposure of the radiation X be stopped is transmitted to the radiation generation device 120 via the wireless communications section 119. In response, the exposure of the radiation X from the radiation source 121 is stopped.

Meanwhile, when the electronic cassette 40 stops the operation for capturing the radiation image, the electronic cassette 40 transmits the image data obtained by the imaging to the console 110.

Accordingly, in step 314, the console 110 waits until the image data is received from the electronic cassette 40. In step 316, image processing is executed to apply the aforementioned missing pixel correction processing to the received image data, and then apply various kinds of correction such as shading correction and the like.

In step 318, the image data to which the image processing has been applied (hereinafter referred to as corrected image data) is stored in the HDD 116. Then, in step 320, the display driver 117 is controlled such that a radiation image represented by the corrected image data is displayed by the display 111 for checking or the like.

In step 322, the corrected image data is transmitted to the RIS server 150 via the hospital internal network 102, after which the present radiation image capture processing program ends. The corrected image data transmitted to the RIS server 150 is stored in the database 150A, and doctors may view the captured radiation image and perform diagnostics and the like.

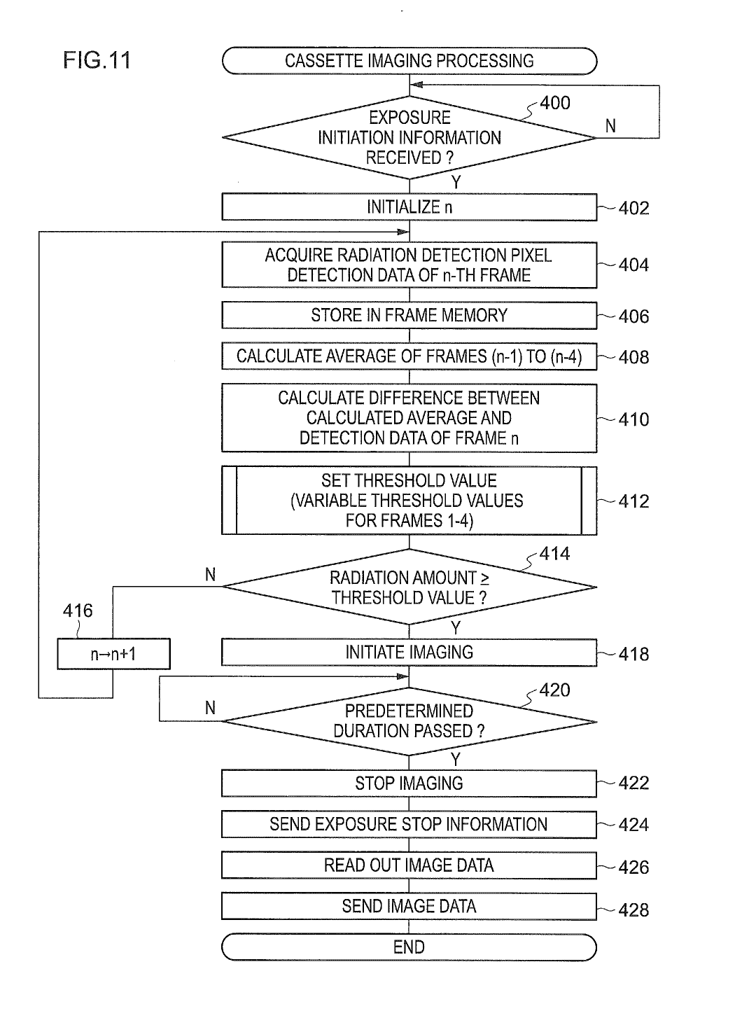

Next, operation of the electronic cassette 40 when the above-described initial information is received from the console 110 is described with reference to FIG. 11. FIG. 11 is a flowchart showing a flow of processing of a cassette imaging processing program that is executed by the CPU 58A of the cassette control section 58 of the electronic cassette 40 at this time. This program is stored in advance in a predetermined region of the memory 58B.

In step 400 of FIG. 11, the cassette control section 58 waits for reception from the console 110 of the above-mentioned instruction information instructing the initiation of exposure. Then, in step 402, a number n representing a count of frames acquired by the detection data acquisition unit 200 is initialized.

In step 404, the gate line driver 52 is controlled so as to turn on the thin film transistors 10 of the radiation detection pixels 32A. Thus, detection results of the radiation detection pixels 32A for an n-th frame are acquired by the functioning of the detection data acquisition unit 200. Then, in step 406, the detection results are stored in the frame memory 202.

In step 408, an average value for frames (n−1) to (n−4) is calculated by the functioning of the average value calculation unit 204. In the present exemplary embodiment, a moving average of the immediately preceding four frames imaged previously is calculated, but this number of frames is not limited to four. Although the first to third frames after the initiation of imaging do not make up four frames, however, average values are calculated for a number of frames are stored in the frame memory 202.

In step 410, a difference between the calculated average value and the detection data of the n-th frame that is acquired is calculated by the functioning of the difference calculation unit 206. Thus, the aforementioned radiation amount information is created. Therefore, signals representing radiation amounts corrected for dark currents may be acquired.

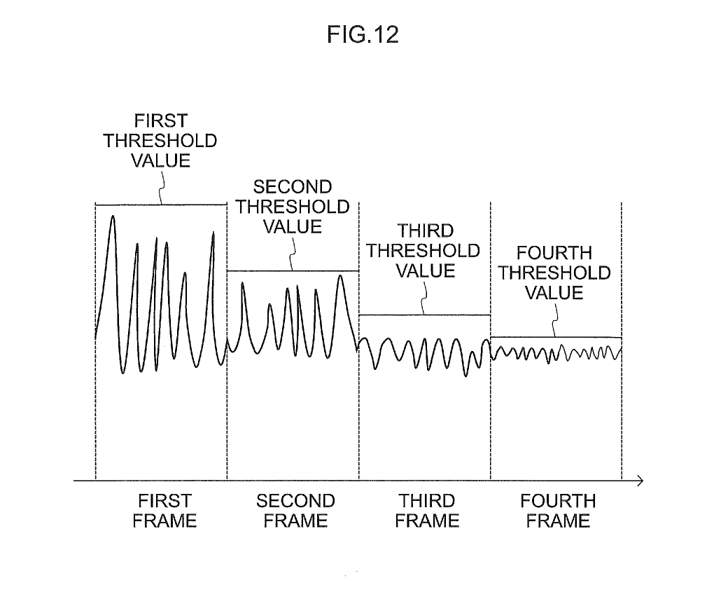

Then, in step 412, threshold value setting processing is carried out. The threshold value setting processing sets a smaller threshold value as larger the number of object frames for calculating an average by the functioning of the average value calculation unit 204 is. Specifically, as shown in FIG. 12, in a case in which the number of object frames for calculating averages is one (the case of a first frame), the possibility that there are still dark currents is high. Therefore, a first threshold value that has been determined in advance to take account of residual dark currents is set. In a case in which the number of object frames is two (the case of a second frame), the residual dark currents are smaller than for the first frame. Therefore, a second threshold value is set, which is smaller than the first threshold value. In a case in which the number of object frames is three (the case of a third frame), the residual dark currents are even smaller than for the second frame. Therefore, a third threshold value is set, which is smaller than the second threshold value. In a case in which the number of object frames is four (cases of a fourth and subsequent frames), the residual dark currents are yet smaller than for the third frame. Therefore, a fourth threshold value is set, which is smaller than the third threshold value. For subsequent frames, dark currents are accounted for by the calculation of the moving average of four frames, and the threshold value is fixed at the fourth threshold value.

When the threshold value is set, in step 414, it is determined whether or not a radiation amount according to the functioning of the radiation detection determination unit 210 is at or above the set threshold value. If the result of the determination is negative, the processing proceeds to step 416. If the result of the determination is affirmative, the exposure of the radiation X from the radiation source 121 is considered to have initiated and the processing proceeds to step 418.

In step 416, n is incremented by 1 to n+1, the processing returns to step 404, and the processing described above is repeated until the exposure of the radiation X is considered to have initiated.

Alternatively, in step 418, charges that have accumulated at the capacitor 9 of each pixel 32 of the radiation detector 20 are discharged, after which the accumulation of charges at the capacitor 9 initiates again, and thus the operation for capturing a radiation image begins.

Then, in step 420, the cassette control section 58 waits for a period specified in advance as a suitable imaging period, in accordance with the imaging target potion, the imaging conditions and the like, to pass. In step 422, the operation for imaging that has been initiated by the processing of step 418 ends. In step 424, the aforementioned exposure stop information is transmitted to the console 110 via the wireless communications section 60.

In step 426, the gate line driver 52 is controlled, On signals are sequentially outputted to the gate lines 34 one line at a time from the gate line driver 52, and the thin film transistors 10 connected to the respective gate lines 34 are sequentially turned on line by line.

When the radiation detector 20 turns on the thin film transistors 10 connected to the gate lines 34 line by line, the charges accumulated in the capacitors 9 flow out into the respective data lines 36 in the form of electronic signals, line by line. The electronic signals flowing into the data lines 36 are converted to digital image data by the first signal processing section 54, and are stored in the image memory 56.

The image data stored in the image memory 56 by step 426 is read out and then, in step 428, the read image data is transmitted to the console 110 via the wireless communications section 60, after which the present cassette imaging processing program ends.

Now, in the electronic cassette 40 according to the present exemplary embodiment, the radiation detector 20 is incorporated such that the radiation X is irradiated thereon from the side thereof at which the TFT substrate 30 is provided.

In a case in which, as shown in FIG. 13, the radiation is irradiated from the side of the radiation detector 20 at which the scintillator 8 is formed and the radiation detector 20 acquires the radiation image with the TFT substrate 30 that is provided at a rear face side relative to the face at which the radiation is incident, which is referred to as penetration side sampling (PSS), light is more strongly emitted from the side of the scintillator 8 that is at the upper face side in FIG. 13 (i.e., to the opposite side thereof from the side at which the TFT substrate 30 is disposed). In a case in which the radiation is irradiated from the side of the radiation detector 20 at which the TFT substrate 30 is fanned and the radiation detector 20 acquires the radiation image with the TFT substrate 30 that is provided at a front face side relative to the face at which the radiation is incident, which is referred to as irradiation side sampling (ISS), radiation that has passed through the TFT substrate 30 is incident on the scintillator 8 and light is more strongly emitted from the side of the scintillator 8 at which the TFT substrate 30 is disposed. Charges are produced by the light emitted from the scintillator 8 to the sensor sections 13 provided at the TFT substrate 30. Therefore, in a case in which the radiation detector 20 is of an ISS type, light emission positions of the scintillator 8 are closer to the TFT substrate 30 than in a case in which the radiation detector 20 is of a PSS type. As a result, the resolution of the radiation images obtained by imaging is higher.

In the radiation detector 20, the photoelectric conversion film 4 is constituted by an organic photoelectric conversion material, and hardly any radiation is absorbed by the photoelectric conversion film 4. Therefore, because amounts of radiation absorbed by the photoelectric conversion film 4 are small even if the radiation is passing through the TFT substrate 30 in accordance with ISS, the radiation detector 20 according to the present exemplary embodiment may suppress a reduction in sensitivity to the radiation. In ISS, the radiation passes through the TFT substrate 30 and reaches the scintillator 8. Thus, in a case in which the photoelectric conversion film 4 of the TFT substrate 30 is constituted by an organic photoelectric conversion material, hardly any radiation is absorbed by the photoelectric conversion film 4 and attenuation of the radiation may be kept low. Therefore, ISS is preferable.

A non-crystalline oxide that constitutes the active layer 17 of each thin film transistor 10, the organic photoelectric conversion material that constitutes the photoelectric conversion film 4, and suchlike are all capable of film formation at low temperatures. Therefore, the substrate 1 may be formed of a plastic resin, aramid or bionanofiber that absorbs small amounts of the radiation. Because radiation absorption amounts of the substrate 1 that is formed thus are small, even in a case in which the radiation passes through the TFT substrate 30 in accordance with ISS, a reduction in sensitivity to the radiation may be suppressed.

As described in detail hereabove, in the present exemplary embodiment, dark currents are corrected for by calculating the moving average of the immediately preceding several frames and calculating a difference between the most recent frame and the calculated average. Therefore, even if there is an abnormality for one frame, because the average of a plural number of frames is calculated, the noise of dark currents may be averaged and eliminated, and effective dark current correction is possible.

Furthermore, in the present exemplary embodiment, the initiation of irradiation of radiation is judged with a threshold value being set in accordance with a number of frames subjected to the averaging calculation when the moving average is calculated. Therefore, the initiation of irradiation of radiation may be detected from when a first frame is acquired.

In the present exemplary embodiment, the dark current noise is large for several frames in an initial period, and as the frame count increases, the dark current noise gets smaller. Therefore, the threshold value is set to be smaller when the number of object frames of the calculation of the moving average is larger. Thus, radiation irradiation initiation detection accuracy for the first several frames may be improved relative to a case in which the threshold value is not changed in gradations.

In the present exemplary embodiment, after a pre-specified frame count, the number of object frames of the calculation of the moving average is set to the immediately preceding several frames. Thus, reliable dark current correction is possible without a processing load for calculating the moving average increasing.

Hereabove, the present invention has been described using the above exemplary embodiment, but the technical scope of the present invention is not to be limited to the scope described in the above exemplary embodiment. Numerous modifications and improvements may be applied to the above exemplary embodiment within a scope not departing from the spirit of the present invention, and modes to which these modifications and/or improvements are applied are to be encompassed by the technical scope of the invention.

Furthermore, the exemplary embodiment described above is not to limit the inventions relating to the claims, and means for achieving the invention are not necessarily to be limited to all of the combination of features described in the exemplary embodiment. Various stages of the invention are included in the above exemplary embodiment, and various inventions may be derived by suitable combinations of the plural structural elements that are disclosed. If some structural element is omitted from the totality of structural elements illustrated in the exemplary embodiment, as long as the effect thereof is provided, a configuration from which the some structural element is omitted may be derived to serve as the invention.

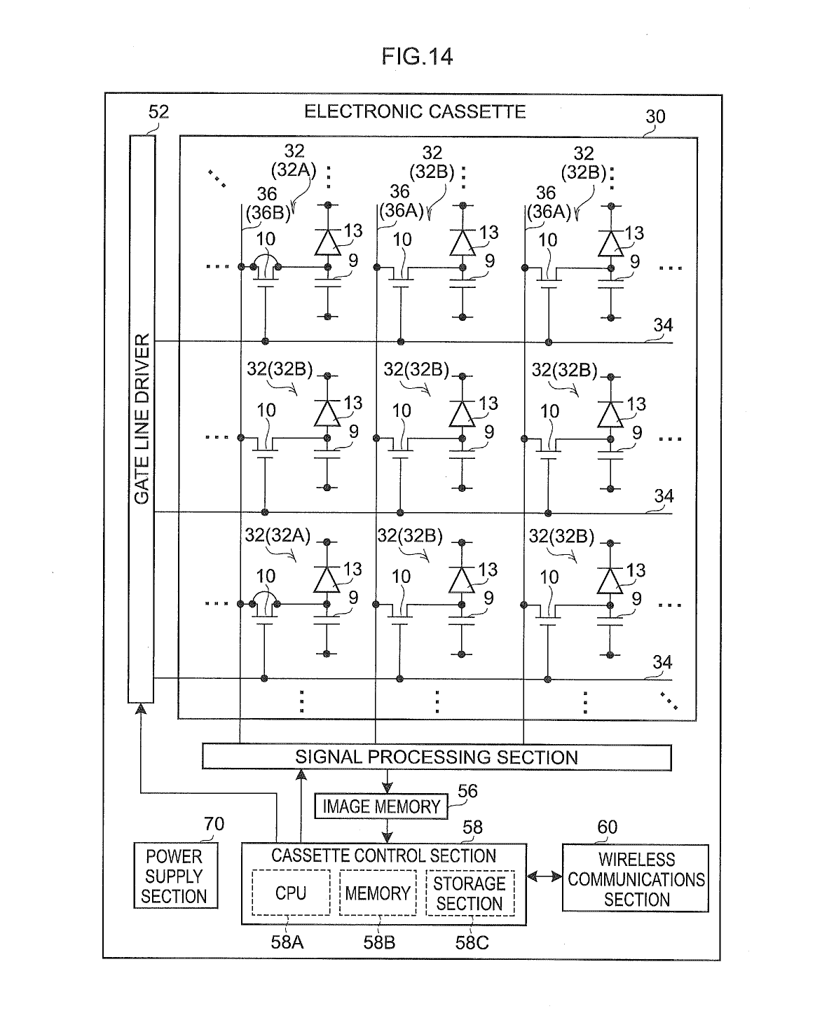

For example, in the exemplary embodiment described above, signals from radiation detection pixels are acquired by the gate line driver 52 being controlled so as to turn on the thin film transistors 10 of the radiation detection pixels 32A. However, a constitution is possible in which a dedicated radiation detection sensor or the like is provided, and a constitution is possible in which, as shown in FIG. 14, the sources and drains of the radiation detection pixels 32A are shorted together. In a case with the structure shown in FIG. 14, charges accumulated at the capacitors 9 of the radiation detection pixels 32A flow into the data lines 36 regardless of the switching states of the thin film transistors 10.

In the case of FIG. 14, a radiation image is captured by the radiation image acquisition pixels 32B of the pixels 32 excluding the radiation detection pixels 32A. Therefore, pixel information of the radiation image may not be acquired for the positions at which the radiation detection pixels 32A are disposed. Accordingly, in the radiation detector 20 according to the present exemplary embodiment, the radiation detection pixels 32A are disposed so as to be scattered, and missing pixel correction processing is executed by the console 110 to generate pixel information of the radiation image for the positions at which the radiation detection pixels 32A are disposed, by interpolation using pixel information obtained by the radiation image acquisition pixels 32B disposed around the radiation detection pixels 32A.

In the exemplary embodiment described above, the moving average of the immediately preceding several frames is calculated by the average value calculation unit 204. However, rather than the moving average, another average value such as an arithmetic mean, a weighted average or the like may be calculated.

In the exemplary embodiment described above, the dark current correction is performed by calculating a difference between the most recent detection data and the average value of the detection data of the immediately preceding several frames. However, a ratio may be found instead of a difference.

The processing illustrated in the flowcharts of the exemplary embodiment described above may be processing that is carried out by hardware, and may be processing that is carried out by software in the form of programs. In a case in which processing is carried out by software in the form of a program, the program may be stored in various kinds of memory medium and distributed.

In the exemplary embodiment described above, a case is described in which an indirect conversion-type device is employed as the radiation image capturing device that is the present invention. However, the present invention is not limited thus, and modes are possible in which the present invention is applied to direct conversion-type devices.

In the exemplary embodiment described above, a case is described in which X-rays are employed as the radiation of the present invention. However, the present invention is not limited thus. For example, other kinds of radiation such as alpha rays, gamma rays or the like may be included.

According to a first aspect of the present invention, there is provided a radiation irradiation initiation determination apparatus including: an acquisition unit that acquires a detection result for each of frames from a detection section that detects radiation; an averaging unit that averages detection results of a plurality of frames which have been previously acquired by the acquisition unit; a calculation unit that calculates at least one of a difference or a ratio between a most recent detection result acquired by the acquisition unit and an averaging result from the averaging unit; and a determination unit that determines whether or not irradiation of radiation has been initiated, on the basis of a calculation result from the calculation unit.

The acquisition unit acquires a detection result for each of frames from the detection section that detects radiation.

The averaging unit averages the detection results from the detection section for a plural number of frames previously acquired by the acquisition unit, and the calculation unit calculates a difference or ratio between the most recent detection result from the detection section acquired by the acquisition unit and the result of averaging by the averaging unit. Thus, dark currents are corrected for.

The determination unit determines whether or not irradiation of radiation has been initiated on the basis of the result of calculation by the calculation unit. For example, the detection unit may judge that irradiation of the radiation has been initiated if the calculation result of the calculation unit is equal to or more than a pre-specified threshold value.

Thus, because a plural number of frames are averaged by the averaging unit and a difference between the most recent frame and the averaging result is calculated, even if there is an abnormality for one frame, dark current noise is averaged and removed by the plural frames being averaged, and effective dark current correction may be carried out.

According to a second aspect of the present invention, the radiation irradiation initiation determination apparatus according to the first aspect may further include a setting unit that sets a threshold value for carrying out the determining by the determination unit, the threshold value being set to a smaller value, the larger that a number of frames that are objects of the averaging by the averaging unit is, wherein the determination unit may determine that irradiation of the radiation has been initiated if the value calculated by the calculation unit is equal to or more than the threshold value set by the setting unit.

Thus, the initiation of irradiation of the radiation may be detected from when the first frame is acquired, by the threshold value being set in accordance with numbers of frames that are objects of the averaging. Radiation irradiation initiation detection accuracy for the first several frames may be improved in comparison with a case in which the threshold value is not changed in gradations.

According to a third aspect of the present invention, the radiation irradiation initiation determination apparatus according to the first aspect may further include a setting unit that sets a threshold value for carrying out the determining by the determination unit to a larger value for frames before dark currents are stable than a pre-specified threshold value for frames when dark currents are stable, wherein the determination unit may determine that irradiation of the radiation has been initiated if the value calculated by the calculation unit is equal to or more than the threshold value set by the setting unit.

Thus, by the threshold value being set to be larger for frames at which detection currents are not stable than for frames at which detection currents are stable, the initiation of irradiation of the radiation may be detected from when the first frame is acquired.

According to a fourth aspect of the present invention, in any one of the first to third aspects, the averaging unit may average signals of a pre-specified number of immediately preceding frames.

Therefore, an increase in a processing load of the averaging unit in association with an increase in the number of frames may be suppressed, and reliable dark current correction may be performed.

According to a fifth aspect of the present invention, in any one of the first to fourth aspects, the detection section may include radiation detection pixels of a radiation detector in which a plurality of radiation image capture pixels and a plurality of the radiation detection pixels are each arranged, the radiation image capture pixels each including a switching element that is set to an On state when charges corresponding to irradiated radiation are to be read out and capturing a radiation image of an imaging subject, and the radiation detection pixels each including the switching element and detecting states of irradiation of the radiation.

According to a sixth aspect of the present invention, in any one of the first to fifth aspects, each radiation detection pixel may include: a conversion section that converts radiation to charges; and the switching element, which is short-circuited between switching terminals.

According to a seventh aspect of the present invention, a radiation image capturing device may include the radiation irradiation initiation determination apparatus according to any one of the first to sixth aspects.

According to an eighth aspect of the present invention, a radiation image capture control apparatus may include the radiation irradiation initiation determination apparatus according to any one of the first to sixth aspects.

According to a ninth aspect of the invention, there is provided a radiation irradiation initiation determination method including: acquiring a detection result for each of frames from a detection section that detects radiation; averaging the previously acquired detection results of a plurality of frames; calculating at least one of a difference or a ratio between a most recent detection result and a result of the averaging; and determining whether or not irradiation of radiation has been initiated on the basis of a result of the calculating.

According to the radiation image capturing method according to the ninth aspect, operation may be the same as in the radiation irradiation initiation determination apparatus according to the first aspect. Thus, similarly to the radiation irradiation initiation determination apparatus according to the first aspect, dark current noise may be averaged and removed, and effective dark current correction may be carried out.

According to a tenth aspect of the present invention, the radiation irradiation initiation determination method according to the ninth aspect may further include setting a threshold value for the determining to a value that is smaller, the larger that a number of frames that are objects of the averaging is, wherein the determining may include determining that irradiation of the radiation has been initiated if the calculated value is equal to or more than the set threshold value.

That is, operation may be the same as in the second aspect of the present invention. Thus, similarly to the second aspect of the present invention, the initiation of irradiation of the radiation may be detected from when the first frame is acquired, and radiation irradiation initiation detection accuracy for the first several frames may be improved.

According to an eleventh aspect of the present invention, the radiation irradiation initiation determination method according to the ninth aspect may further include setting a threshold value for the determining to a larger value for frames before dark currents are stable than a pre-specified threshold value for frames when dark currents are stable, wherein the determining may include determining that irradiation of the radiation has been initiated if the calculated value is equal to or more than the set threshold value.

That is, operation may be the same as in the third aspect of the present invention. Thus, similarly to the third aspect of the present invention, the initiation of irradiation of the radiation may be detected from when the first frame is acquired.

According to a twelfth aspect of the present invention, in any one of the ninth to eleventh aspects, the averaging may include averaging signals of a pre-specified number of immediately preceding frames.

That is, operation may be the same as in the fourth aspect of the present invention. Thus, similarly to the fourth aspect of the present invention, an increase in a processing load of the averaging may be suppressed, and reliable dark current correction may be performed.

According to a thirteenth aspect of the invention, there is provided a non-transitory computer readable medium storing a program causing a computer to execute a radiation irradiation initiation determination processing, the processing including: acquiring a detection result for each of frames from a detection section that detects radiation; averaging the previously acquired detection results of a plurality of frames; calculating at least one of a difference or a ratio between a most recent detection result and a result of the averaging; and determining whether or not irradiation of radiation has been initiated on the basis of a result of the calculating.

According to the radiation irradiation initiation determination program recited by the thirteenth aspect of the present invention, operation may be the same as in the radiation irradiation initiation determination apparatus according to the first aspect of the present invention. Thus, similarly to the radiation irradiation initiation determination apparatus according to the first aspect of the invention, dark current noise may be averaged and removed, and effective dark current correction may be carried out.

According to a fourteenth aspect of the present invention, in the thirteenth aspect, the processing may further include setting a threshold value for the determining to a value that is smaller, the larger that a number of frames that are objects of the averaging is, wherein the determining may include determining that irradiation of the radiation has been initiated if the calculated value is equal to or more than the set threshold value.

That is, operation may be the same as in the second aspect of the present invention. Thus, similarly to the second aspect of the present invention, the initiation of irradiation of the radiation may be detected from when the first frame is acquired, and radiation irradiation initiation detection accuracy for the first several frames may be improved.

According to a fifteenth aspect of the present invention, in the thirteenth aspect, the processing may further include setting a threshold value for the determining to a larger value for frames before dark currents are stable than a pre-specified threshold value for frames when dark currents are stable, wherein the determining may include determining that irradiation of the radiation has been initiated if the calculated value is equal to or more than the set threshold value.

That is, operation may be the same as in the third aspect of the present invention. Thus, similarly to the third aspect of the present invention, the initiation of irradiation of the radiation may be detected from when the first frame is acquired.

According to a sixteenth aspect of the present invention, in any one of the thirteenth to fifteenth aspects, the averaging may include averaging signals of a pre-specified number of immediately preceding frames.

That is, operation may be the same as in the fourth aspect of the present invention. Thus, similarly to the fourth aspect of the present invention, an increase in a processing load of the averaging may be suppressed, and reliable dark current correction may be performed.

According to the present invention, plural frames are averaged and differences or ratios between the most recent frames and the averaging results are calculated. Thus, even if there is an abnormality for one frame, because a plural number of frames are averaged, dark current noise may be averaged and eliminated, and effective dark current correction may be carried out when detecting for the initiation of irradiation of radiation.

Embodiments of the present invention are described above, but the present invention is not limited to the embodiments as will be clear to those skilled in the art.

What is claimed is:

1. A radiation irradiation initiation determination apparatus comprising:
an acquisition unit that acquires a detection result for each of frames from a detection section that detects radiation;
an averaging unit that averages detection results of a plurality of frames which have been previously acquired by the acquisition unit;
a calculation unit that calculates at least one of a difference or a ratio between a most recent detection result acquired by the acquisition unit and an averaging result from the averaging unit; and
a determination unit that determines whether or not irradiation of radiation has been initiated, on the basis of a calculation result from the calculation unit.

2. The radiation irradiation initiation determination apparatus according to claim 1, further comprising a setting unit that sets a threshold value for carrying out the determining by the determination unit, the threshold value being set to a smaller value, the larger that a number of frames that are objects of the averaging by the averaging unit is,
wherein the determination unit determines that irradiation of the radiation has been initiated if the value calculated by the calculation unit is equal to or more than the threshold value set by the setting unit.

3. The radiation irradiation initiation determination apparatus according to claim 1, further comprising a setting unit that sets a threshold value for carrying out the determining by the determination unit to a larger value for frames before dark currents are stable than a pre-specified threshold value for frames when dark currents are stable,
wherein the determination unit determines that irradiation of the radiation has been initiated if the value calculated by the calculation unit is equal to or more than the threshold value set by the setting unit.

4. The radiation irradiation initiation determination apparatus according to claim 1, wherein the averaging unit averages signals of a pre-specified number of immediately preceding frames.

5. The radiation irradiation initiation determination apparatus according to claim 1, wherein the detection section includes radiation detection pixels of a radiation detector in which a plurality of radiation image capture pixels and a plurality of the radiation detection pixels are each arranged, the radiation image capture pixels each including a switching element that is set to an On state when charges corresponding to irradiated radiation are to be read out and capturing a radiation image of an imaging subject, and the radiation detection pixels each including the switching element and detecting states of irradiation of the radiation.

6. The radiation irradiation initiation determination apparatus according to claim 5, wherein each radiation detection pixel includes: a conversion section that converts radiation to charges; and the switching element, which is short-circuited between switching terminals.

7. A radiation image capturing device comprising:
the radiation irradiation initiation determination apparatus according to claim 1.

8. A radiation image capture control apparatus comprising:
the radiation irradiation initiation determination apparatus according to claim 1.

9. A radiation irradiation initiation determination method comprising:
acquiring a detection result for each of frames from a detection section that detects radiation;
averaging the previously acquired detection results of a plurality of frames;
calculating at least one of a difference or a ratio between a most recent detection result and a result of the averaging; and
determining whether or not irradiation of radiation has been initiated on the basis of a result of the calculating.

10. The radiation irradiation initiation determination method according to claim 9, further comprising setting a threshold value for the determining to a value that is smaller, the larger that a number of frames that are objects of the averaging is,
wherein the determining includes determining that irradiation of the radiation has been initiated if the calculated value is equal to or more than the set threshold value.

11. The radiation irradiation initiation determination method according to claim 9, further comprising setting a threshold value for the determining to a larger value for frames before dark currents are stable than a pre-specified threshold value for frames when dark currents are stable,
wherein the determining includes determining that irradiation of the radiation has been initiated if the calculated value is equal to or more than the set threshold value.

12. The radiation irradiation initiation determination method according to claim 9, wherein the averaging includes averaging signals of a pre-specified number of immediately preceding frames.

13. A non-transitory computer readable medium storing a program causing a computer to execute radiation irradiation initiation determination processing, the processing comprising:
acquiring a detection result for each of frames from a detection section that detects radiation;
averaging the previously acquired detection results of a plurality of frames;
calculating at least one of a difference or a ratio between a most recent detection result and a result of the averaging; and
determining whether or not irradiation of radiation has been initiated on the basis of a result of the calculating.

14. The computer readable medium according to claim 13, wherein the processing further comprises setting a threshold value for the determining to a value that is smaller, the larger that a number of frames that are objects of the averaging is,
wherein the determining includes determining that irradiation of the radiation has been initiated if the calculated value is equal to or more than the set threshold value.

15. The computer readable medium according to claim 13, wherein the processing further comprising setting a threshold value for the determining to a larger value for frames before dark currents are stable than a pre-specified threshold value for frames when dark currents are stable,
wherein the determining includes determining that irradiation of the radiation has been initiated if the calculated value is equal to or more than the set threshold value.

16. The computer readable medium according to claim 13, wherein the averaging includes averaging signals of a pre-specified number of immediately preceding frames.
